(19) United States
(12) Patent Application Publication
(10) Pub. No.: US 2016/0199754 A1
(43) Pub. Date: Jul. 14, 2016

(54) PROCESS FOR SEPARATING A DIVINYL HYDROCARBON FROM MONOVINYL HYDROCARBONS AND/OR NON-VINYL COMPOUNDS

(71) Applicant: BLUE CUBE IP LLC, Midland, MI (US)

(72) Inventors: Megan E. Donaldson, Linwood, MI (US); Robert M. Campbell, Midland, MI (US); Bruce S. Holden, Midland, MI (US); Timothy C. Frank, Midland, MI (US); Bruce D. Hook, Lake Jackson, TX (US)

(73) Assignee: Blue Cube IP LLC, Midland, MI (US)

(21) Appl. No.: 14/913,617

(22) PCT Filed: Aug. 27, 2014
(86) PCT No.: PCT/US2014/052835

§ 371 Date: Feb. 22, 2016

Related U.S. Application Data
(60) Provisional application No. 61/871,375, filed on Aug. 29, 2013.

(51) Int. Cl. B01D 15/38; C07C 7/12

(52) U.S. Cl. CPC B01D 15/38; C07C 7/12; B01D 2015/3838

(57) ABSTRACT

A process for separating a divinyl hydrocarbon from a composition mixture stream containing at least a divinyl hydrocarbon and other components, the process including (a) passing a composition mixture feed stream containing at least (i) a divinyl hydrocarbon; and (ii) a monovinyl hydrocarbon, and/or (iii) a non-vinyl hydrocarbon through a process-scale chromatography unit; wherein the process scale chromatography unit includes a ligand exchange media comprising a metal adapted to form a ligand with an olefin functionality; wherein the metal of the ligand exchange media is loaded on an adsorbent; and wherein the divinyl hydrocarbon is adsorbed onto the ligand exchange media; (b) passing a weak first elution solvent through the unit to elute the monovinyl hydrocarbon and/or the non-vinyl hydrocarbon from the unit; (c) passing a strong second elution solvent through the unit to elute the divinyl hydrocarbon product stream from the unit; and (d) recovering the divinyl hydrocarbon product stream having been separated from the other components in the composition mixture stream; and an apparatus therefor.

### FIELD

The present invention is related to a process and apparatus for purifying a divinyl hydrocarbon product including separating impurities from the product. More specifically, the present invention is related to a process and apparatus for separating a divinyl hydrocarbon from monovinyl hydrocarbons and/or other impurities such as non-vinyl compounds via a process utilizing ligand exchange process scale chromatography.

### BACKGROUND

A divinyl hydrocarbon such as divinylbenzene (DVB) is an important raw material used in the production of other important downstream products. Downstream products made from for example a divinyl hydrocarbon such as DVB are beneficially used in many applications including for example coatings, composites, laminates and adhesives.

Typically, during the production of DVB, a crude DVB material has to undergo a separation process, to separate a desired DVB product from undesired byproducts or impurities such as ethylvinylbenzene (EVB) and diethylbenzene (DEB) that may be present in the crude DVB material. Currently a conventional distillation process is practiced in the art as the separation process. The separation process is preferred to produce a DVB raw material which is substantially free of the above undesired byproducts and impurities, i.e., a pure DVB raw material.

By increasing the purity of DVB, less waste is produced resulting in a more efficient process. In addition, by separating and isolating an undesired byproduct such as EVB, the EVB can be recycled back to a DVB reactor, thus improving the yield of the DVB process. Thus, a pure DVB product is desirable for use in downstream products.

Some research efforts have explored the use of silver or copper coordination to separate saturated and unsaturated compounds. For example, U.S. Patent Application Publication No. 20100093530 discloses a method of preparing a composition using argentation chromatography. The above publication describes a process to separate saturated and unsaturated compounds using simulated moving bed chromatography with an adsorbent modified with silver (argentized). The preferred embodiment of the above publication involves carbon chains from 10 to 30 with the primary focus towards separating fatty acid methyl esters (FAME). The above publication does not disclose the separation of aromatic structures such as DVB and EVB.

U.S. Pat. No. 3,217,052 discloses the separation of DVB from a mixture containing DVB and EVB. The above patent describes a method of using solid copper or silver salts in a DVB/EVB solution to coordinate preferentially with DVB, allowing for filtration of the DVB/salt compound and then recovery of the product via heating. However, the method described in the above patent suffers from the following disadvantages: (1) produces high levels of solids and having to handle such solids, (2) loses product in the wet cake, (3) requires having to deal with waste disposal of the salt cake, (4) provides a limited capacity for DVB, less than 50% at temperatures above 4° C., since the coordinated compound requires separation and then treatment to recover the DVB, (5) forms tar upon heating the solids, and (6) does not provide the ability to remove insoluble tars from the solids, leading to fouling and short metal lifetimes.

A review article published by Eldridge, Safarik, Olefin/Paraffin Separations by Reactive Absorption: A Review, Ind. Eng. Chem. Res., 1998, 37, 2571-2581; describes reactive absorption methods for separating olefin/paraffin mixtures, primarily using complexation with solutions of silver or copper salts in a reactive absorber column followed by passing the solution through a stripper to recover the absorption solution. These methods are typically applied to gas streams, such as those of ethane/ethylene.

### SUMMARY

Process-scale chromatography is a technique used in the process of the present invention for separating various desired components such as DVB from undesired impurities such as EVB. Process-scale chromatography can improve the separation process and reduce costs associated with capital and energy consumption through reductions in capital and energy usage beyond those associated with conventional distillation. The process of the present invention improves the separation significantly, enabling potential reductions in capital requirements and enhanced product purity beyond prior art current limitations. The present invention includes the use of a ligand exchange process-scale chromatography as the separation mechanism and an apparatus therefor. Process-scale chromatography allows a divinyl hydrocarbon such as DVB to weakly complex and then desorb with a metal-modified adsorbent, potentially enabling a higher throughput with reduced processing.

One aspect of the present invention includes a process for separating a divinyl hydrocarbon from a composition mixture stream containing at least a divinyl hydrocarbon and other components. For example, the process includes the steps of:

(a) passing a composition mixture feed stream containing at least (i) a divinyl hydrocarbon; and (ii) a monovinyl hydrocarbon, and/or (iii) a non-vinyl hydrocarbon through a process-scale chromatography unit with or without heating; wherein the process scale chromatography unit includes a ligand exchange media comprising a metal adapted to form a ligand with an olefin functionality; wherein the metal of the ligand exchange media is loaded on an adsorbent; and wherein the divinyl hydrocarbon is adsorbed onto the ligand exchange media;

(b) passing a weak first elution solvent through the unit to elute the monovinyl hydrocarbon and/or the non-vinyl hydrocarbon from the unit;

(c) passing a strong second elution solvent through the unit to elute the divinyl hydrocarbon product stream from the unit; and

(d) recovering the divinyl hydrocarbon product stream having been separated from the other components in the composition mixture stream.

One preferred embodiment of the present invention includes, for example, using the above process for separating divinylarene from a composition mixture stream containing at least an ethylvinylarene, a diethylarene and a divinylarene.

Another aspect of the present invention includes an apparatus for carrying out the above process.

### BRIEF DESCRIPTION OF THE DRAWINGS

For the purpose of illustrating the present invention, the drawings show a form of the present invention which is presently preferred. However, it should be understood that the present invention is not limited to the embodiments shown in the drawings.

FIG. 1 is a schematic block flow diagram showing a process of the present invention.

FIG. 2 is a graphical illustration of one embodiment of the present invention showing a chromatogram with results of DVB separated from EVB using a silver-loaded column in a process-scale chromatography unit.

FIG. 3 is a graphical illustration showing a chromatogram with results of DVB separated from EVB using a silica column in a process-scale chromatography unit as a comparison to the chromatogram of FIG. 2.

### DETAILED DESCRIPTION

The process of the present invention uses a process scale chromatography unit to separate a feed stream fed into the unit into two or more streams for example an extract as a product stream and a raffinate as an undesired impurity stream. Examples of process scale chromatography units include one or more chromatographic columns packed with separation media. The different feed stream components can have different affinities to the stationary phase leading to different migration velocities through the chromatography unit. This leads to the separation of the feed stream components.

The separation mechanism of the present invention may involve the use of a metal ion ligand exchange chromatography such as wherein the metal ion can be silver or copper ion. Ligand exchange chromatography allows for low temperature separation, and thus low energy usage for the separation. For example, the separation mechanism uses silver or copper ion complexation or coordination with an olefinic organic species to form an organometallic complex to separate the components.

Divinyl hydrocarbons such as DVB have a greater affinity for silver or copper ions on a stationary phase, enabling greater retention of a divinyl hydrocarbon over monovinyl and/or non-vinyl hydrocarbons. Thereby, the process scale chromatography, such as simulated moving bed (SMB) chromatography, may be used in a continuous process to generate and separate a divinyl hydrocarbon-rich product stream as a slow eluting extract stream from a by-product-rich stream as a fast eluting raffinate stream.

Some of the advantages of the present invention over current conventional distillation practice include for example reduced energy usage with a resulting reduced carbon footprint, reduced byproduct formation from oligomerization of divinyl hydrocarbons and/or monovinyl hydrocarbons in reboilers of distillation towers resulting in improved product yields, and smaller distillation towers than in the conventional distillation processes of divinyl hydrocarbons resulting in lower predicted capital costs.

One of the important advantages of the present invention includes a process having a lower cost to produce a very high purity divinyl hydrocarbon such as DVB (e.g. greater than 95%); as opposed to using a conventional distillation process which provides for example low yields, high tar formation, and low tower life.

One broad embodiment of the present invention is directed to a process for separating a divinyl hydrocarbon from other impurities including the steps of:

(a) passing a divinyl hydrocarbon stream containing impurities through process scale chromatography using ligand exchange media;

(b) separating the divinyl hydrocarbon stream as an extract stream exiting from the process scale chromatography from the impurities stream as a raffinate stream exiting from the process scale chromatography; and

(c) recovering the divinyl hydrocarbon stream separated from the impurities stream.

Another broad embodiment of the present invention is directed to a process for separating a divinyl hydrocarbon from a composition mixture stream containing at least a divinyl hydrocarbon and other components. For example, the process in this embodiment includes the steps of:

(a) passing a composition mixture feed stream containing at least (i) a divinyl hydrocarbon; and (ii) a monovinyl hydrocarbon, and/or (iii) a non-vinyl hydrocarbon through a process-scale chromatography unit with or without heating; wherein the process scale chromatography unit includes a ligand exchange media comprising a metal adapted to form a ligand with an olefin functionality; wherein the metal of the ligand exchange media is loaded on an adsorbent; and wherein the divinyl hydrocarbon is adsorbed onto the ligand exchange media;

(b) passing a weak first elution solvent through the unit to elute the monovinyl hydrocarbon and/or the non-vinyl hydrocarbon from the unit;

(c) passing a strong second elution solvent through the unit to elute the divinyl hydrocarbon product stream from the unit; and

(d) recovering the divinyl hydrocarbon product stream having been separated from the other components in the composition mixture stream.

The composition feed stream that is fed through a process-scale chromatography unit such as a SMB using ligand exchange media, can contain at least one divinyl hydrocarbon, at least one monovinyl hydrocarbon, and at least one non-vinyl hydrocarbon. In one embodiment, the feed stream of the present invention may be sourced, for example, from a divinylarene production process. For example, the effluent from a dehydrogenation reactor in a divinylarene production unit may be used as the feed stream. In another embodiment, the feed stream may be sourced from a volatile hydrocarbon removal distillation operation such as the distillation process that follows the above dehydrogenation reactor.

The composition mixture feed stream used in step (a) preferably contains at least one divinyl hydrocarbon. The divinyl hydrocarbon may include for example a divinylarene such as divinylbenzene (DVB); or a mixture of two or more divinylarenes. An arene herein is any molecule containing at least one aromatic ring. The divinyl hydrocarbon useful in the present invention can also include non-aromatic olefinic cyclic compounds such as divinylcyclohexane. Thus, while the product includes impurities that may be derivatives or isomers and most of which can be aromatic, i.e. arenes, the product can contain methyl, hydroxyl, cyclobutyl or other groups or moieties.

The concentration of the components in the feed stream and the product stream can be measured by gas chromatography. More importantly the product purity is measured to meet product specifications which determine product performance in enduse applications. As an illustration, when the divinyl hydrocarbon is DVB, the DVB purity generally can be as low as about 40 weight percent (wt %) and as high as about 95 wt % in one embodiment; and some instances can also be even greater than 95 wt % in another embodiment. In still another embodiment for example, the DVB purity can be in the range between about 40 wt % and about 80 wt %; and between about 55 wt % and about 65 wt % in yet another embodiment.

Generally, the amount of the divinyl hydrocarbon present in the composition mixture feed stream may be for example, from 10 wt % to about 50 wt % in one embodiment, from about 20 wt % to about 40 wt % in another embodiment; from about 25 wt % to about 35 wt % in still another embodiment; and from about 28 wt % to about 32 wt % in yet another embodiment.

The composition mixture feed stream used in step (a) preferably contains at least one monovinyl hydrocarbon. The monovinyl hydrocarbon includes for example an ethylvinylarene such as EVB, vinyltoluene, styrene and mixtures thereof.

Generally, the amount of the monovinyl hydrocarbon present in the composition may be for example, from 10 wt % to about 50 wt % in one embodiment, from about 20 wt % to about 40 wt % in another embodiment; from about 26 wt % to about 36 wt % in still another embodiment; and from about 28 wt % to about 32 wt % in yet another embodiment.

The composition mixture feed stream used in step (a) may optionally contain at least one non-vinyl hydrocarbon. The non-vinyl hydrocarbon includes for example a diethylarene such as DEB, ethyltoluene, toluene, benzene, naphthalene, and mixtures thereof.

Generally, the amount of the non-vinyl hydrocarbon present in the composition may be for example, from 5 wt % to about 50 wt % in one embodiment, from about 15 wt % to about 42 wt % in another embodiment; from about 26 wt % to about 38 wt % in still another embodiment; and from about 28 wt % to about 32 wt % in yet another embodiment.

In step (a) of the present invention process, the feed stream mixture is preferably passed through a process-scale chromatography unit without heating. When heated, the feed stream can be heated up to a temperature of about 100° C. in one embodiment, from about 10° C. to about 60° C. in another embodiment; and from about 20° C. to about 40° C. in still another embodiment.

The process scale chromatography unit useful in the present invention process may include a ligand exchange media adapted to form a ligand with an olefin functionality such as DVB. For example, the media may include a metal, metal salts, metal or metal salts deposited on a solid support, or other material capable of adsorbing the divinyl hydrocarbon onto the ligand exchange media or forming a ligand with the divinyl hydrocarbon. “Media” herein means a solid packing within the separation column or other solid separation means within the separation column of the process scale chromatography unit.

The metal of the ligand exchange media is preferably loaded on an adsorbent. The adsorbents useful in the present invention may include, for example, cation exchange resins, silica, zeolites, alumina, and mixtures thereof. The adsorbent may be modified with the metal through various means including for example, but not limited to, mechanical and electrostatic means as known in the art. In one preferred embodiment, the ligand exchange media can include for example silver or copper modified adsorbents. The ligand exchange media can also include other metal-modified adsorbents such as platinum, palladium, gold, ruthenium, mercury, iridium, any metal known to be active to form complexes with organic compounds, or mixtures thereof.

In step (a) of the present invention process, the divinyl hydrocarbon is preferably adsorbed onto the ligand exchange media. Then, in step (b) a weak first elution solvent is passed through the process-scale chromatography unit to elute from the unit, the monovinyl hydrocarbon and/or the non-vinyl hydrocarbon present in the feed mixture stream. The stream of the monovinyl hydrocarbon and/or the non-vinyl hydrocarbon exiting the unit is the raffinate.

A “weak elution solvent” herein comprises a solvent or a solvent mixture that is capable of eluting monovinyl hydrocarbon species such as an ethylvinylarene and/or a diethylarene species; and other non-vinyl hydrocarbon species from a process scale chromatography unit without eluting divinyl hydrocarbon species such as a divinylarene species.

Examples of weak elution solvents useful in the present invention include, but are not limited to, hexane, heptane, cyclohexane, isooctane, aliphatic hydrocarbons, carbon dioxide (e.g., under supercritical process conditions), or mixtures thereof.

After step (b), a strong second elution solvent in step (c) is passed through the process-scale chromatography unit to elute from the unit, at least some of the divinyl hydrocarbon product that had formed a ligand with an olefin functionality of the divinyl hydrocarbon product via the ligand exchange media, and thus forming a divinyl hydrocarbon product stream as the extract exiting the unit.

A “strong elution solvent” herein comprises a solvent or solvent mixture that is capable of solvating and eluting divinyl hydrocarbon species such as a divinylarene or any olefinic species that is complexed to the metal modified media from a process scale chromatography unit. The strong elution solvent useful in the present invention may include, for example, one or more of organic solvents such as ethers, ketones, esters, alcohols, aromatics, chlorinated solvents, and mixtures thereof.

In one embodiment, the elution solvent useful in the present invention may include a solvent or solvent mixture that is capable of eluting divinyl hydrocarbons such as a divinylarene or an olefinic species that is complexed to the metal modified media used in the process. Examples of strong second elution solvents useful in the present invention include, but are not limited to, acetone, acetonitrile, methyl tert-butylether, ethyl acetate, tetrahydrofuran, methanol, ethanol, isopropanol, water, or mixtures thereof.

The process includes a recovery step (d) wherein the divinyl hydrocarbon, product stream having been separated from the other components in the composition mixture stream, is recovered by various known processes including for example, distillation, liquid-liquid extraction, decantation, and combinations thereof.

One embodiment of the recovery step (d) for recovering the divinyl hydrocarbon separated from the monovinyl hydrocarbons can include any conventional recovery process such as for example a distillation method and apparati therefor. Conventional distillation can be used to isolate the divinyl hydrocarbon product from the elution solvent and allow for recovery and recycle of the solvent back to the process scale chromatography unit for increased efficiency. When a distillation operation is used to recover the divinyl hydrocarbon product from the elution solvent, an elution solvent with a higher boiling point than the divinyl hydrocarbon product is one preferred embodiment. For example, the elution solvent used is preferably one that has a normal boiling point greater than about 200° C. in the case wherein the divinyl hydrocarbon is divinylbenzene.

For example, when a divinylarene such as divinylbenzene (DVB) is processed herein, the use of a higher boiling point solvent allows the DVB to be recovered immediately following the feed stream passing through the process scale chromatography unit (e.g., SMB).

Advantageously, the DVB is recovered as a purified overhead product in the distillation process free from heavies such as inhibitors, heavy impurities, oligomers, and tars with relatively low concentrations of DVB in the reboiler where degradation can occur. Then, a subsequent distillation of a bottoms stream containing solvent would separate the elution solvent from heavier components for recycle of the solvent.

In another embodiment, an elution solvent with a boiling point lower than that of the divinyl hydrocarbon product can also be used. For example, the elution solvent used is preferably one that has a normal boiling point lower than about 200° C. in the case wherein the divinyl hydrocarbon is divinylbenzene. In such embodiment, DVB and heavies would be taken as a bottoms product in the first distillation with light solvent taken overhead for recycle, and the separation of DVB from heavies would follow, for example, in a subsequent distillation operation.

An optional step of the process of the present invention includes recovering the elution solvent from the raffinate stream. Generally, any conventional recovery process can be used such as for example a distillation method and apparatus therefor. Conventional distillation can be used to recover the elution solvent from the raffinate stream and allow for recovery and recycle of the solvent back to the process scale chromatography unit for increased efficiency. The isolated raffinate stream can also be recycled internally to improve process yields.

The overall process of the present invention, using process-scale chromatography, may be carried out at a predetermined temperature, a predetermined pressure, and for a predetermined period of time sufficient to separate for example divinyl hydrocarbons such as divinylarene from other monovinyl hydrocarbons such as ethylvinylarene and diethylarene. The conditions of the process may vary depending on the raw materials used in the process and the type of column used in the process-scale chromatography unit.

For example, the temperature of the process may be generally from about 0° C. to about 100° C. in one embodiment; from about 10° C. to about 60° C. in another embodiment; and from about 20° C. to about 40° C. in still another embodiment.

Generally, the pressure of the process may be from about 1 atm to about 20 atm in one embodiment, between about 1.5 atm to about 10 atm in another embodiment, and between about 2 atm to about 5 atm in still another embodiment.

In addition, the process of present invention, and/or any of the steps thereof, may be carried out in a batch, a semi-batch or a continuous process. In order to carry out the process, a novel apparatus is used in the above process as described herein. Thus, another broad embodiment of the present invention is directed to an apparatus for continuously separating a divinyl hydrocarbon from a mixture containing the divinyl hydrocarbon, a monovinyl hydrocarbon and a non-vinyl hydrocarbon.

The following disclosure describes one non-limiting preferred embodiment of the process of the present invention, wherein the process includes separating a divinylarene such as divinylbenzene (DVB) from other components in a feed stream for example a monovinylarene such as ethylvinylarene and more specifically such as ethylvinylbenzene (EVB) and diethylarene such as diethylbenzene (DEB). For example, a feed stream containing divinylbenzene, ethylvinylbenzene and diethylbenzene can be passed through process scale chromatography unit using a ligand exchange media; and then the divinylbenzene separated from the ethylvinylbenzene and diethylbenzene can be recovered by known recovery means.

In addition, the feed stream may contain various isomers of the components in the feed stream. For example, the divinylarene in the feed stream composition may include for example, ortho, meta, and para isomers or any combination thereof; the ethylvinylarene in the above feed stream composition may include for example, ortho, meta, and para isomers or any combination thereof; and the diethylarene in the feed stream composition may include for example, ortho, meta, and para isomers or any combination thereof.

In generally, the concentrations of the components present in the feed stream of the above preferred embodiment can vary depending on the components in the feed stream composition. For example in one embodiment, the amount of divinylarene in the composition may be from about 10 wt % to about 50 wt %; the amount of ethylvinylarene in the composition may be from 10 wt % to about 50 wt %; the amount of diethylarene in the composition may be from 5 wt % to about 50 wt %; and the amount of other impurities, such as non-vinyl hydrocarbons in the composition may be from 0 wt % to about 20 wt %.

In another embodiment, the composition feed stream to the chromatography process may be, for example, a crude DVB mixture produced by the dehydrogenation reactor process; or the composition feed stream used in the present invention can originate from a bottoms stream that originates from a lights removal distillation tower operation which removes water and light organics from the crude DVB mixture in a similar fashion as in a conventional distillation process. If the feed stream to the chromatography process is a crude DVB mixture, the non-removed lights and water would can be removed from the eluted EVB/DEB stream from the chromatography process prior to recovering the EVB/DEB from the solvent for recycle to the dehydrogenation reactors.

The separation process of the present invention in the above embodiment can provide for example a DVB product having several improved properties such as for example product purity. Typically, using conventional processes, DVB product is usually produced at concentrations of 55 wt %, 63 wt %, and 80 wt % with the balance being primarily EVB. Using the separation process of the present invention, on the other hand, higher concentrations of DVB product can be produced such as for example 95 wt % or more. The conventional distillation processes may be able to produce high concentrations of product, but this is typically done at the cost of increased yield loss to oligomer formation due to higher processing temperatures and increased retention times in the distillation train of the conventional process. The present invention would avoid this source of yield loss through moderate processing temperatures and process retention times.

The apparatus of the present invention, in one preferred embodiment generally includes a chromatographic separation process. The chromatographic separation process can be carried out in batch, semi-batch, or continuous modes. One preferred method for carrying out the chromatographic separation process is continuous due to improved process efficiency. The preferred continuous method uses simulated moving bed technology in which the process simulates the countercurrent flow of a liquid phase, consisting of “feed” and “desorbent”, and a solid adsorbent phase.

For example, the process-scale chromatography unit useful in the present invention may include a conventional simulated moving bed (SMB) process which includes one or more columns connected in series and packed with chromatographic resin to enable the separation. The process-scale chromatography unit generates a “raffinate” stream containing the fast eluting component(s) with low affinity for the resin and an “extract” stream that contains the slow eluting component(s) with higher affinity for the resin. The “desorbent” or the “eluent” is added to the process to aid in removal of the high affinity component(s) from the resin. During the process, the SMB unit switches the input streams and the output streams of liquids from one column to another (or between column sections) to approach the theoretical performance of a true countercurrent solid-liquid flow. Switching the input streams and the output streams from one column to another can be accomplished using valves (e.g., rotary valves or a network of two-position or multi-position valves) which work in conjunction with the inlet and outlet lines of the multiple fixed-bed columns. The liquid flow rates of the feed streams and the step times for the valves of the SMB unit are controlled so that the slow and fast eluting components move in opposite directions relative to the movement or switching of inlet and outlet ports of the apparatus.

In a preferred embodiment, the fixed-bed columns of the SMB unit are configured to provide four zones to separate the feed stream into two fractions: (1) the extract, which includes the slow-eluting fraction; and (2) the raffinate, which includes the fast-eluting fraction. The four zones of the SMB unit each perform a different function. For example, Zone I contains fixed-bed columns between the eluent inlet and the extract outlet; Zone II contains fixed-bed columns between the extract outlet and the feed inlet; Zone III contains fixed-bed columns between the feed inlet and the raffinate outlet; and Zone IV contains fixed-bed columns between the raffinate outlet and the eluent inlet. Within the SMB unit, Zones II and III serve to allow the fast and slow components to move farther apart, while Zones I and IV serve to prevent the slow components from falling too far back and the fast components from moving too far forward, respectively.

In one embodiment, the feed stream can be separated into multiple product streams to enable ternary separation to produce a divinyl hydrocarbon product stream such as a divinylarene, a monovinyl hydrocarbon stream such as an ethylvinylarene, and/or a diethylarene product stream. When third and subsequent product streams are desired to be isolated, the product streams from the chromatography unit can be recovered by employing a multi-component SMB separation scheme.

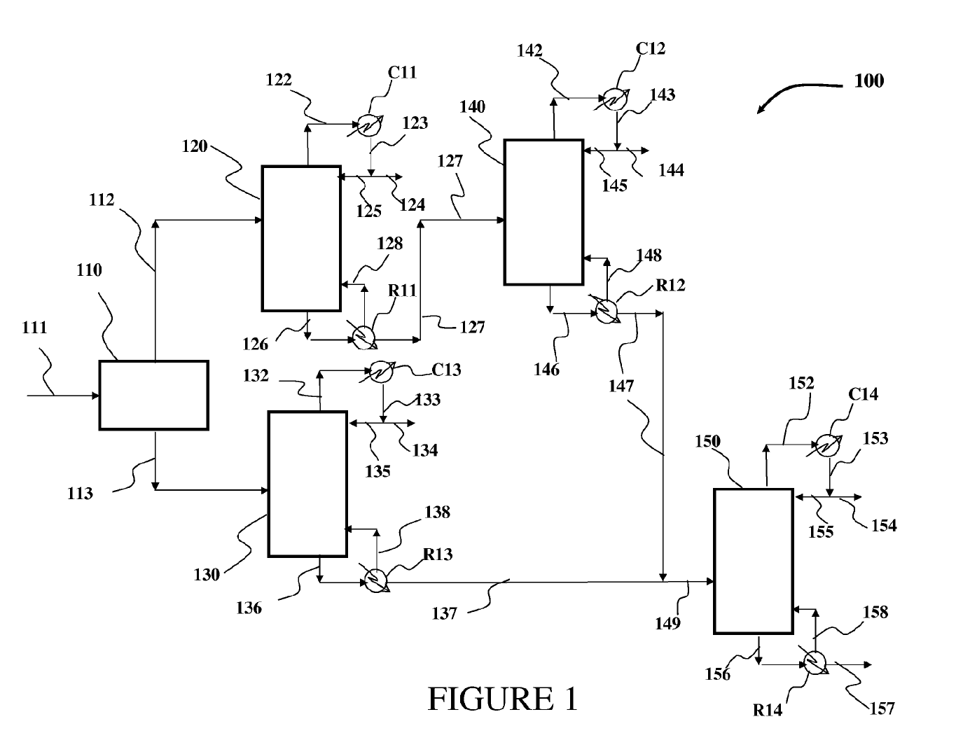

With reference to FIG. 1, there is shown one embodiment of the present invention process generally indicated by numeral 100, which includes an SMB system 110. In addition, the process of FIG. 1 includes tower 120, 130, 140 and 150 in fluid communication with each other to process the streams from the SMB. The raffinate stream 112 containing EVB, lights and solvent for example is passed through columns 120 and 140 for further processing; and the extract stream 113 containing DVB and solvent for example is passed through columns 130 and 150 for further processing.

The raffinate stream 112 from SMB 110 is processed through column 120, where the lights stream as overhead stream 122 is separated from an EVB and solvent stream which exits column 120 as bottoms stream 126. The lights stream 122 is passed through a condenser C11 and at least a portion of the lights stream 123 is removed from column 120 as stream 124 and at least a portion is recycled to column 120 as stream 125. The bottoms stream 126 is passed through a reboiler R11 and at least a portion of the bottoms stream 126 is removed from column 120 as stream 127 and at least a portion is recycled to column 120 as stream 128. Stream 127 leaving column 120 containing EVB and solvent becomes the feed stream to column 140 for further processing.

The feed stream 127 is processed in column 140, where the EVB stream as overhead stream 142 is separated from the solvent stream which exits column 140 as bottoms stream 146. The EVB stream 142 is passed through a condenser C12 and at least a portion of the EVB stream 143 is removed from column 140 as stream 144 and at least a portion is recycled to column 140 as stream 145. The bottoms stream 146 is passed through a reboiler R12 and at least a portion of the bottoms stream 146 is removed from column 140 as stream 147 and at least a portion is recycled to column 140 as stream 148. Stream 147 leaving column 140 containing solvent is combined with the bottoms stream 137 from column 130 to form the feed stream 149 to column 150 for further processing.

The extract stream 113 from SMB 110 is processed through column 130, where the DVB stream as overhead stream 132 is separated from the solvent stream which exits column 130 as bottoms stream 136. The DVB stream 132 is passed through a condenser C13 and at least a portion of the DVB stream 133 is removed from column 130 as stream 134 and at least a portion is recycled to column 130 as stream 135. Stream 134 is the DVB product recovered from the present invention process.

The bottoms stream 136 is passed through a reboiler R13 and at least a portion of the bottoms stream 136 is removed from column 130 as stream 137 and at least a portion is recycled to column 130 as stream 138. Stream 137 leaving column 130 containing solvent is combined with the bottoms stream 147 from column 140 to form the feed stream 149 to column 150 for further processing.

The combined solvent stream 149 becomes the feed stream to column 150 and is processed in column 150 to remove/separate heavies present in the solvent stream 149 from the solvent stream. In column 150, a solvent stream exits column 150 as overhead stream 152 which is separated from the heavies stream which exits column 150 as bottoms stream 156. The solvent stream 152 is passed through a condenser C14 and at least a portion of the solvent stream 153 is removed from column 150 as stream 154 and at least a portion is recycled to column 150 as stream 155. The bottoms stream 156 is passed through a reboiler R14 and at least a portion of the bottoms stream 156 is removed from column 150 as stream 157 and at least a portion is recycled to column 150 as stream 158. Stream 157 leaving column 150 containing heavies can be sent to other unit operations for further processing or can be sent to storage. Stream 154 leaving column 150 containing solvent can be sent to other unit operations for further processing, recycled back to the one or more unit operations of the present invention process, or can be sent to storage.

### EXAMPLES

The following examples and comparative examples further illustrate the present invention in detail but are not to be construed to limit the scope thereof.

Various terms and designations used in the following examples are explained herein below:

“UV” stands for ultraviolet.

Chromspher 5 LIPIDS is a silver loaded ion exchange column commercially available from Agilent Technologies.

IPA:MTBE is the ratio of isopropanol to methyl tertiary butyl ether; and the material is commercially available from Thermo Fisher Scientific.

Zorbax Rx-Sil is a silica HPLC column commercially available from Agilent Technologies.

### Example 1

### DVB Separation Results on Silver-Loaded Column

In this Example 1, the separation of divinylarene from ethylvinylarene was demonstrated with laboratory-scale liquid chromatography experiments. More specifically, in this Example 1 divinylbenzene (DVB) was separated from ethylvinylbenzene (EVB) and diethylbenzene (DEB) through injection of a solution mixture onto a silver-loaded liquid chromatography column. The following chromatography conditions were used:
Column: Chromspher 5 LIPIDS 250×4.6 mm, 5 um (Varian/Agilent), Cat. No. CP28313
Flow: 1 mL/min
Temperature: ambient (about 25° C.)
Mobile phase A: hexane
Mobile phase B: 10:90 IPA:MTBE

Gradient:

| Time | Composition |
|---|---|
| 0.0 | 2% B |
| 2.0 | 2% B |
| 30.0 | 35% B |
| 35.0 | 35% B |

Detection: UV at 254 nm
Injection volume: 10 μL
Sample preparation: 5 mg/mL in hexane

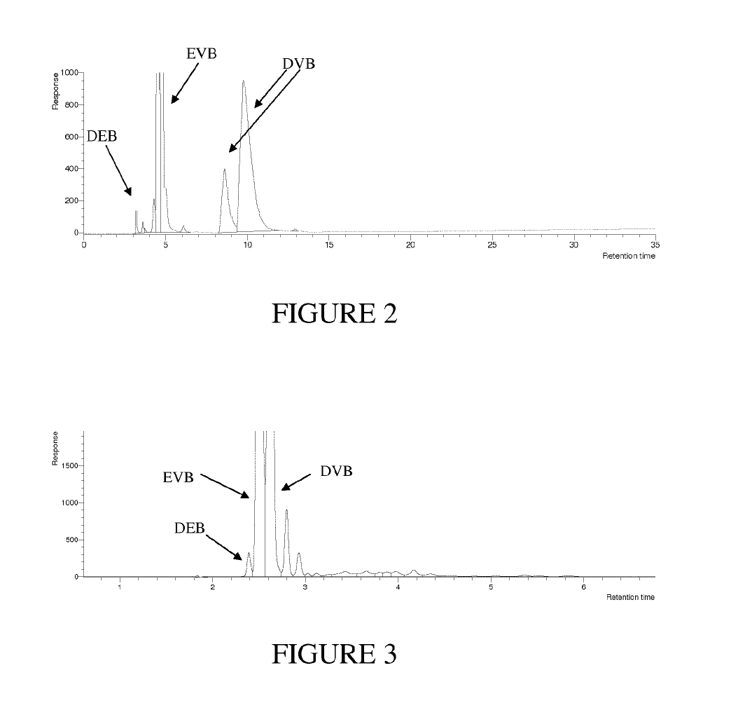

As shown in the chromatogram of FIG. 2, the silver-loaded column demonstrated excellent separation of the DVB synthetic mixture. The chromatogram shows elution of the species over 8 minutes with retention times of 3 minutes for DEB, 5 minutes for EVB, and between 8 and 11 minutes for DVB.

As previously discussed, the separation mechanism involves complexation between the silver ions and the vinyl functionality on the molecules. The components elute in order of increasing vinyl functionality, thereby supporting the hypothesis that metal-vinyl coordination is the dominant separation mechanism. Unexpectedly, the laboratory-scale experiment of this Example 1 indicated not only significant DVB/EVB separation, but also separation of the meta- and para-DVB isomers.

The present invention process improves performance through use of ligand exchange media that enhances the separation of mixtures containing varying levels of unsaturation.

### Comparative Example A

### DVB Separation Results on Silica Column

In this Comparative Example A, the separation of divinylarene from ethylvinylarene was evaluated with laboratory-scale liquid chromatography experiments using conventional media. More specifically, in this Comparative Example A divinylbenzene (DVB) was separated from ethylvinylbenzene (EVB) and diethylbenzene (DEB) through injection of a solution mixture onto a normal phase silica liquid chromatography column. The following chromatography conditions were used:
Column: Zorbax Rx-Sil, 250×4.6 mm, 5 μm
Flow: 1.5 mL/minute
Temperature: ambient
Mobile phase: hexane
Detection: UV at 254 nm
Injection volume: 5 μL
Sample prep: 20 mg/mL in heptanes

As shown in the chromatogram of FIG. 3, the normal phase silica evaluation showed marginal resolution of DEB, EVB, and DVB. This conventional media technique primarily utilizes polarity to drive the separation of the various species. Although the results show peak separation, the molecules all elute within one minute, which indicates that the polarity differences are relatively slight. It is expected that the above separation using conventional media will only become poorer with the use of larger, process-scale media and loadings. Thus, conventional, polarity-driven separation mechanisms are not expected to provide good performance for process-scale chromatography of DVB/EVB mixtures.

1. A process for separating a divinyl hydrocarbon from a composition mixture stream containing at least a divinyl hydrocarbon and other components, the process comprising the steps of:
(a) passing a composition mixture feed stream containing at least (i) a divinyl hydrocarbon; and (ii) a monovinyl hydrocarbon, and/or (iii) a non-vinyl hydrocarbon through a process-scale chromatography unit with or without heating; wherein the process scale chromatography unit includes a ligand exchange media comprising a metal adapted to form a ligand with an olefin functionality; wherein the metal of the ligand exchange media is loaded on an adsorbent; and wherein the divinyl hydrocarbon is adsorbed onto the ligand exchange media;
(b) passing a weak first elution solvent through the unit to elute the monovinyl hydrocarbon and/or the non-vinyl hydrocarbon from the unit;
(c) passing a strong second elution solvent through the unit to elute the divinyl hydrocarbon product stream from the unit; and
(d) recovering the divinyl hydrocarbon product stream having been separated from the other components in the composition mixture stream.

2. The process of claim 1, wherein the divinyl hydrocarbon is a divinylarene, a divinylcycloaliphatic, a divinylaliphatic having carbon atoms of greater than 4, a polyvinyl hydrocarbon, an acetylenic hydrocarbon, or mixtures thereof.

3. The process of claim 2, wherein the divinylarene is divinylbenzene.

4. The process of claim 1, wherein the monovinyl hydrocarbon is an ethylvinylarene, and wherein the ethylvinylarene is ethylvinylbenzene.

5. The process of claim 1, wherein mixture feed stream contains naphthalene, an ethylvinylarene, a diethylarene, and light components.

6. The process of claim 1, wherein the non-vinyl hydrocarbon is a diethylarene and wherein the diethylarene is diethylbenzene.

7. The process of claim 1, including the step of recovering the monovinyl hydrocarbon having been separated from the composition mixture stream; and wherein the monovinyl hydrocarbon is an ethylvinylarene, a diethylarene, or a mixture thereof.

8. The process of claim 1, wherein step (a) is carried out without heating.

9. The process of claim 1, wherein the metal of the ligand exchange media is loaded onto an adsorbent mechanically, or wherein the metal of the ligand exchange media is loaded onto an adsorbent electrostatically.

10. The process of claim 1, wherein the solvent used in step (b) and step (c) is the same solvent, and wherein the solvent is acetone, acetonitrile, ethyl acetate, tetrahydrofuran, methanol, ethanol, isopropanol, water, or mixtures thereof.

11. The process of claim 1, wherein the metal of the ligand exchange media is silver, copper, salts of silver, salts of copper, platinum, palladium, gold, ruthenium, mercury, iridium, any metal known to be active to form complexes with organic compounds, or mixtures thereof.

12. The process of claim 11, wherein the silver or copper is disposed on a support material.

13. The process of claim 1, wherein the ligand exchange media is a silver-modified adsorbent, a copper-modified adsorbent, or mixtures thereof.

14. The process of claim 1, wherein the metal of the ligand exchange media is regenerated.

15. The process of claim 1, wherein the composition mixture stream includes further (iv) a solvent and wherein the solvent is higher boiling point than divinylarene.

16. The process of claim 1, wherein the composition mixture stream includes further (iv) a solvent and wherein the solvent is lower boiling point than divinylarene.

17. The process of claim 1, wherein the composition of the composition mixture stream comprises from about 10 weight percent to about 50 weight percent divinyl hydrocarbon; from about 5 weight percent to about 50 weight percent monovinyl hydrocarbon, and from about 0 weight percent to about 20 weight percent non-vinyl hydrocarbon.

18. The process of claim 1, wherein the composition of the divinylarene product stream on a solvent-free basis comprises from about 50 weight percent to about 96 weight percent divinyl hydrocarbon; about 0 weight percent to about 20 weight percent from about 4 weight percent to about 50 weight percent monovinyl hydrocarbon; and from about 0 weight percent to about 20 weight percent non-vinyl hydrocarbon.

19. The process of claim 1, wherein the monovinyl hydrocarbon in the composition mixture stream comprises meta isomers, para isomers, ortho isomers or any combination thereof; wherein the non-vinyl hydrocarbon in the composition mixture stream comprises meta isomers, para isomers, ortho isomers, or any combination thereof; and
wherein the divinyl hydrocarbon in the composition mixture stream comprises meta isomers, para isomers, ortho isomers, or any combination thereof.

20. The process of claim 19, including the steps of:
(i) separating at least a portion of one or more of the meta isomer, para isomer, ortho isomer, or combination thereof from the other isomers and/or other components in the product stream; and
(ii) adjusting the meta/para isomer ratio of the divinyl hydrocarbon in the composition mixture stream to a meta/para isomer ratio of from about 0.5 to about 4.0.

21. An apparatus for separating a divinyl hydrocarbon from a composition mixture stream containing at least a divinyl hydrocarbon and other components, the apparatus comprising:
(A) a means for passing a composition mixture feed stream containing at least
(i) a divinyl hydrocarbon; and
(ii) a monovinyl hydrocarbon, and/or
(iii) a non-vinyl hydrocarbon through a process-scale chromatography unit with or without heating; wherein the process scale chromatography unit includes a ligand exchange media comprising a metal adapted to form a ligand with an olefin functionality; wherein the metal of the ligand exchange media is loaded on an adsorbent; and wherein the ligand exchange media is adapted for adsorbing the divinyl hydrocarbon onto the ligand exchange media;
(b) a means for passing a weak first elution solvent through the unit to elute the monovinyl hydrocarbon and/or the non-vinyl hydrocarbon from the unit;
(c) a means for passing a strong second elution solvent through the unit to elute the divinyl hydrocarbon product stream from the unit; and
(d) a means for recovering the divinyl hydrocarbon product stream having been separated from the other components in the composition mixture stream.
